(19) United States
(12) Patent Application Publication
(10) Pub. No.: US 2013/0303818 A1
(43) Pub. Date: Nov. 14, 2013

(54) METHOD FOR PRODUCING ALPHA-OLEFIN UNSATURATED DIMER

(71) Applicants: Junichi Inagaki, Ichihara-shi (JP); Masashi Machida, Ichihara-shi (JP); Masaki Okano, Ichihara-shi (JP); Minako Tsuji, Ichihara-shi (JP)

(72) Inventors: Junichi Inagaki, Ichihara-shi (JP); Masashi Machida, Ichihara-shi (JP); Masaki Okano, Ichihara-shi (JP); Minako Tsuji, Ichihara-shi (JP)

(73) Assignee: IDEMITSU KOSAN CO., LTD., Tokyo (JP)

(21) Appl. No.: 13/997,865

(22) PCT Filed: Jan. 11, 2012
(86) PCT No.: PCT/JP2012/000098

§ 371 Date: Jun. 25, 2013

(30) Foreign Application Priority Data
Jan. 13, 2011 (JP) 2011-004912

(51) Int. Cl. C07C 2/34

(52) U.S. Cl. CPC C07C 2/34
USPC 585/511

(57) ABSTRACT

A method for producing an unsaturated dimer by reacting an α-olefin having 6 to 14 carbon atoms in an agitated vessel, wherein a metallocene-based catalyst is used as a reaction catalyst; and the supply flow rate of hydrogen (V_{H}) to the vessel is 0.2 to 1.2 [(NL/hr) per L of olefin] and the agitation power (Pv) is 0.18 KW/m³ or more of the time of reaction.

### TECHNICAL FIELD

The invention relates to a method for producing an α-olefin unsaturated dimer.

### BACKGROUND ART

A poly α-olefin (PAO) obtained by polymerizing 1-octene, 1-decene and 1-dodecene is used in the field of a lubricant oil base of an engine or a transmitter, a viscosity index improver, an antiwear additive, a friction modifier, ink, paint, emulsion and a mold releasing agent for toner, or the like.

PAO has several viscosity levels which are required according to application. As for the method for controlling viscosity, a method in which a polymer with a specific polymerization degree can be selectively obtained by selecting a polymerization catalyst or a method in which fractions differing in polymerization degree are separated after polymerization.

On the other hand, a dimer of α-olefin can be produced in the same manner as in the case of PAO, and is used for raw materials of a plasticizer of a resin or the like. Since it is used as a raw material, it is preferred that a dimer of α-olefin be a dimer having a reactive group (a vinylidene group). Therefore, as the standard for judging the quality thereof, the remaining ratio of an unsaturated bond (vinylidene) is used.

As the method for producing a PAO, Patent Documents 1 to 3 each discloses a method in which an α-olefin is polymerized by using a metallocene catalyst, for example. In the method disclosed in these documents, hydrogen is supplied during the reaction in order to enhance the activity of the metallocene catalyst. In addition to the effect of enhancing the catalytic activity, hydrogen also has an effect of converting the unsaturated bond of the resulting polymer to a single bond. The above-mentioned publications mainly refer to a method for producing a PAO to be used for lubrication oil. Reducing the unsaturated bond of the polymer is preferable in respect of improving thermal stability of lubricant oil.

On the other hand, in the production of an unsaturated dimer of an α-olefin, use of hydrogen is not necessarily preferable since hydrogen lowers the remaining ratio of an unsaturated bond. However, there is a problem that, without using hydrogen, the activity of the metallocene is deteriorated to lower the reaction activity.

### RELATED ART DOCUMENTS

### Patent Documents

Patent Document 1: JP-A-2001-335607
Patent Document 2: WO2010/074233
Patent Document 3: WO2010/053022

### SUMMARY OF THE INVENTION

The invention is aimed at providing a method for producing an α-olefin unsaturated dimer in a high yield at a sufficiently high reaction rate on the industrial scale.

As a result of intensive studies, the inventors have found that, in the polymerization reaction of an α-olefin, by controlling the hydrogen supply flow rate (V_{H}) and the agitation power (Pv) to a specific level, a desired dimer can be produced in a high yield at a high reaction rate. The invention has been made on this finding.

According to the invention, the following method for producing an unsaturated dimer of an α-olefin is provided.

1. A method for producing an unsaturated dimer by reacting an α-olefin having 6 to 14 carbon atoms in an agitated vessel, wherein a metallocene-based catalyst is used as a reaction catalyst; and the supply flow rate of hydrogen (V_{H}) to the vessel is 0.2 to 1.2 [(NL/hr) per L of olefin] and the agitation power (Pv) is 0.18 KW/m³ or more of the time of reaction.
2. The method for producing an unsaturated dimer according to 1, wherein the supply flow rate of hydrogen (V_{H}) is 0.2 to 0.8 [(NL/hr) per L of olefin] and the agitation power (Pv) is set to 0.7 KW/m³ or more.
3. The method for producing an unsaturated dimer according to 1 or 2, wherein the α-olefin is selected from 1-octene, 1-decene and 1-dodecene.
4. The method for producing an unsaturated dimer according to 1 or 2, wherein the α-olefin is 1-decene.

According to the production method of the invention, an unsaturated dimer of an α-olefin can be produced efficiently in a high yield.

### MODE FOR CARRYING OUT THE INVENTION

In the method for producing an unsaturated dimer of an α-olefin of the invention, in an agitated vessel, α-olefins having 6 to 14 carbon atoms are allowed to react with each other while stirring, thereby to produce a dimer.

Examples of an α-olefin having 6 to 14 carbon atoms include 1-hexene, 1-octene, 1-decene, 1-dodecene and 1-tetradecene. Preferable α-olefins include 1-octene, 1-decene and 1-dodecene, with 1-decene being particularly preferable.

The α-olefin may be used in combination of two or more or may be used singly.

In the invention, a metallocene-based catalyst is used as the reaction catalyst. As the metallocene-based catalyst used in the invention, a catalyst disclosed in WO2010/053022 (Patent Document 3) can be preferably used.

Specifically, a metallocene-based catalyst containing a transition metal compound represented by the following formula (1) and an aluminum-containing co-catalyst (B) is used in the invention.

{C₅R¹R²R³R⁴(A¹R^{a}R^{b}R^{c})}{C₅R⁵R⁶R⁷R⁸(A²R^{d}R^{e}R^{f})}MXY (1)

wherein R¹ to R⁸, (A¹R^{a}R^{b}R^{c}) and (A²R^{d}R^{e}R^{f}) are independently a substituent which is bonded to a cyclopentadienyl group. R¹ to R⁸ are independently a hydrogen atom or a hydrocarbon group having 1 to 10 carbon atoms; and R^{a} to R^{f} are independently a hydrocarbon group having 1 to 10 carbon atoms. Two or more groups selected from R^{a}, R^{b} and R^{C} may be bonded with each other to form a ring; two or more groups selected from R^{d}, R^{e} and R^{f} may be bonded with each other to form a ring; A¹ and A² are independently an element belonging to the 14^{th} group of the periodic table of the elements; M is a transitional metal belonging to the 4^{th} group of the periodic table of elements; and X and Y are independently a covalent ligand or an ionic ligand.

Further, the metallocene-based catalyst is a metallocene-based catalyst containing a transitional metal compound represented by the following formula (2) and methylaluminoxane.

(RC₅H₄)₂MX₂ (2)

wherein R is a hydrogen atom or a hydrocarbon group having 1 to 10 carbon atoms, M is a transitional metal belonging to the 4^{th} group of the periodic table of elements; and X is a covalent ligand or an ionic ligand.

Of the transitional metal compounds represented by the above formulas (1) and (2), bis-t-butylcylopentadienylzirconium dichloride is particularly preferable.

As the co-catalyst (B) containing aluminum, methylaluminoxane is preferable.

By allowing an α-olefin to be reacted by using the above-mentioned metallocene-based catalyst, a mixture containing a component larger than a timer in addition to a dimer is obtained. Therefore, in the invention, a reaction product is fractionated by distillation or the like, whereby dimer components are recovered.

In the invention, no particular restrictions are imposed on the polymerization method. Any of bulk polymerization, solution polymerization, suspension polymerization, slurry polymerization, vapor phase polymerization or the like can be used. As for polymerization conditions, the polymerization temperature is normally 0 to 200° C., preferably 30 to 150° C., more preferably 40 to 120° C. As for the amount ratio of the raw material olefin to the catalyst, it is preferred that the raw material olefin/the transition metal compound (molar ratio) be 1 to 10⁸, particularly 100 to 10⁵. The polymerization time is normally 5 minutes to 20 hours, the reaction pressure is preferably from normal pressure to 0.2 MPaG, with from normal pressure to 0.1 MPaG being particularly preferable.

In the production method of the invention, it is preferred that production be conducted without using a solvent. However, a solvent may be used. In this case, aromatic hydrocarbons such as benzene, toluene, xylene and ethylbenzene; alicyclic hydrocarbons such as cyclopentane, cyclohexane and methylcyclohexane; aliphatic hydrocarbons such as pentane, hexane, heptane and octane and halogenated hydrocarbons such as chloroform and dichloromethane can be used. These solvents may be used alone or in combination of two or more.

In the invention, a preliminary polymerization can be conducted by using a polymerization catalyst. A preliminary polymerization can be conducted by allowing a small amount of olefin to contact the catalyst components. No specific restrictions are imposed on this method, and a known method can be used. No specific restrictions are imposed on an olefin used in a preliminary polymerization. For example, ethylene, an α-olefin having 3 to 20 carbon atoms or a mixture thereof can be given. It is advantageous to use the same olefin as that of a raw olefin used in this polymerization. A preliminary polymerization temperature is normally −20 to 200° C., preferably −10 to 130° C., and more preferably 0 to 80° C. In the preliminary polymerization, as a solvent, inactive hydrocarbons, aliphatic hydrocarbons, aromatic hydrocarbons, monomers or the like can be used. Of these, aliphatic hydrocarbons and aromatic hydrocarbons are preferable.

Further, a preliminary polymerization may be conducted without using a solvent. In the preliminary polymerization, it is preferable to adjust the conditions such that the amount of a preliminary polymerization product per mill mole of transition metal components in a catalyst becomes 1 to 10,000 g, in particular, 1 to 1,000 g.

In the production method of the invention, when a raw olefin is reacted, catalytic activity is improved by supplying hydrogen in the reaction system, whereby the reaction rate (k) is increased. The supply flow rate (V_{H}) of hydrogen to a vessel during the reaction is 0.2 to 1.2 [(NL/hr per L of olefin)], preferably 0.3 to 1.0 [(NL/hr per L of olefin)], and particularly preferably 0.7 to 0.8 [(NL/hr per L of olefin)]. If V_{H} is smaller than 0.2 [(NL/hr) per L of olefin], the catalyst activity is not improved, resulting in a lowering of the reaction rate. On the other hand, if V_{H} is larger than 1.2 [(NL/hr per L of olefin)], the amount of hydrogen in the vessel becomes excessive, and the double bond of a dimer is hydrogenated irrespective of the agitation power.

The unit means the amount of hydrogen supplied (normal liter) per hour relative to per liter (L) of an olefin as a raw material.

In the invention, the agitation power (Pv) of an agitator is 0.18 KW/m³ or more, preferably 0.3 KW/m³ or more, particularly preferably 0.4 KW/m³ or more. If Pv is smaller than 0.18 KW/m³, catalytic activity may not be improved since hydrogen cannot contact the metallocene catalyst sufficiently.

The upper limit of Pv is not particularly restricted. However, taking the scale of equipment and the size of apparatus into consideration, the upper limit of Pv is normally around 2 KW/m³ or less. It is preferred that Pv be 1.5 KW/m³ or less, with 1.2 KW/m³ being particularly preferable.

The agitation power indicates the power of agitation, and is defined by the agitation power per unit volume (the volume of raw olefin in the reaction chamber).

No specific restrictions are imposed on the vessel or the agitator used in the invention. A reaction vessel or an agitated vessel which is known in this technical field can be used. The supply flow rate of hydrogen (gas flow rate) is preferably 2.5 m/s or more. The supply flow rate of hydrogen can be controlled by adjusting the supply amount or by adjusting the aperture of a hydrogen supply mouth (nozzle or the like).

As the agitation blade, a Pfaudler impeller, a max blend impeller, a turbine blade or the like are preferable.

### EXAMPLES

### Example 1

A 25,000 L-reaction vessel provided with a turbine blade as the agitator was used. To the reaction vessel, 15,000 L of 1-decene as raw olefin was supplied. Stirring was conducted for 30 minutes with bubbling with N₂.

Next, the pressure inside the vessel was increased to 12 kPaG with N₂. The pressure was further increased by 2 kPaG with H₂.

Heat medium oil was circulated in a heating jacket, whereby the temperature of 1-decene in the reaction chamber was increased to 40° C. and kept constant.

To the reaction vessel, methylaluminoxane (MAO) as the co-catalyst and bis-t-butylcyclopentadienylzirconium dichloride (t-BuCp)₂ZrCl₂ (hereinafter referred to as T2) was added. MAO was added in an amount of 4000 μmol/(per L of 1-decene) and T2 was added in an amount of 40 μmol/(per L of 1-decene). The reaction temperature was 40° C., the agitation power was 0.30 kW/m³ and the hydrogen flow was 0.20 [(NL/hr/per L of 1-decene].

Thereafter, a sample was recovered every hour to calculate the reaction rate. The reaction time was 7.0 hours.

### Comparative Example 1

A dimer of 1-decene was produced in the same manner as in Example 1, except that the agitation power and the hydrogen flow were changed to the values shown in Table 1.

The reaction rate constant k and the vinylidene purity under the conditions in Example 1 and Comparative Example 1 were measured. The results are shown below.

TABLE 1

| | Agitation | Hydrogen | Reaction rate | |
|---|---|---|---|---|
| | power | flow | constant k | Vinylidene purity |
| | P_{V} | V_{H} | (min⁻¹) | ( %) |
| Example1 | 0.30 | 0.20 | 0.0068 | 91.4 |
| Com. Ex. 1 | 0.30 | 0.01 | 0.0013 | 95.3 |

P_{V}: [kW/m³]
V_{H}: [(NL/hr) per L of 1-decene]

Reaction rate constant k

The reaction rate constant k was obtained by the following formula:

k=−ln(C/C₀)/t

(wherein k is a reaction rate constant, C is a raw material concentration after the reaction, C₀ is a raw material concentration before the reaction, and t is a reaction time)

Vinylidene purity

Measured by gas chromatography (GC main body: GC2010, manufactured by Shimadzu Corporation). The conditions are as follows.
(1) Column: Ultra 2, 25 m×0.2 mm×0.33 μm, MAX. 325° C.

### (2) Flow

Import pressure: 207.5 kPa

Column flow: 1.37 ml/min

Linear velocity: 41.7 cm/s

Split ratio: 30.0

Total flow: 47.4 ml/min

Injection mode: SPLIT

Control mode: Linear velocity

Carrier gas: He

FID/INJ=300° C./300° C.

Column: Temperature programmed meter 42 mim

Injection amount: 1 μl

Temperature elevation pattern: Hold at 100° C. for 1 minute, and elevated at a rate of 10° C. per minute for 20 minutes to be 300° C., followed by measurement at 300° C.

### INDUSTRIAL APPLICABILITY

The invention is suitable as the method for producing an unsaturated dimer of an α-olefin.

Although only some exemplary embodiments and/or examples of this invention have been described in detail above, those skilled in the art will readily appreciate that many modifications are possible in the exemplary embodiments and/or examples without materially departing from the novel teachings and advantages of this invention.

Accordingly, all such modifications are intended to be included within the scope of this invention.

The documents described in the specification are incorporated herein by reference in its entirety.

1: A method for producing an unsaturated dimer, comprising:
reacting an α-olefin having 6 to 14 carbon atoms in an agitated vessel with a reaction catalyst,
wherein the reaction catalyst is a metallocene-based catalyst, a supply flow rate of hydrogen (V_{H}) to the agitated vessel is 0.2 to 1.2 (NL/hr) per L of the α-olefin, and an agitation power (Pv) is 0.18 KW/m³ or more of a reaction time.

2: The method according to claim 1,
wherein the supply flow rate (V_{H}) is 0.2 to 0.8 (NL/hr) per L of the α-olefin and the agitation power (Pv) is set to 0.7 KW/m³ or more.

3: The method according to claim 1,
wherein the α-olefin is selected from the group consisting of 1-octene, 1-decene, and 1-dodecene.

4: The method according to claim 1, wherein the α-olefin is 1-decene.

5: The method according to claim 2,
wherein the α-olefin is selected from the group consisting of 1-octene, 1-decene, and 1-dodecene.

6: The method according to claim 2,
wherein the α-olefin is 1-decene.
